(19) United States
(12) Patent Application Publication
(10) Pub. No.: US 2025/0163129 A1
(43) Pub. Date: May 22, 2025

(54) COMBINATION MEDICAMENTS COMPRISING HLA FUSION PROTEINS

(71) Applicant: IMMUNOS THERAPEUTICS AG, Schlieren (CH)

(72) Inventors: Osiris MARROQUIN BELAUNZARAN, Schlieren (CH); Marco GUALANDI, Schlieren (CH); Anahita RAFIEI, Schlieren (CH)

(73) Assignee: IMMUNOS THERAPEUTICS AG, Schlieren (CH)

(21) Appl. No.: 18/681,074

(22) PCT Filed: Aug. 5, 2022
(86) PCT No.: PCT/EP2022/072133

§ 371 Date: Feb. 5, 2024

(30) Foreign Application Priority Data
Aug. 5, 2021 (EP) 21190003.0

(51) Int. Cl. C07K 14/74; A61K 38/00; C07K 16/28

(52) U.S. Cl. CPC C07K 14/70539; C07K 16/2803; C07K 16/2896; A61K 38/00; C07K 2319/30

(57) ABSTRACT

The present invention relates to combination medicaments comprising (human leukocyte antigen) soluble HLA heavy chain polypeptides, and an inhibitor of the interaction between CD47 and signal regulatory protein alpha (SIRP□□□□for use in treating cancer. Further aspects of the invention relate to an inhibitor of the interaction between CD47 and SIRP□, for use in patients receiving treatment with a soluble HLA heavy chain polypeptide according to the invention.

### CROSS-REFERENCE TO RELATED APPLICATION

This is the U.S. National Stage of International Patent Application No. PCT/EP2022/072133 filed on Aug. 5, 2022, which claims the priority of European Patent Application No.EP21190003.0, filed Aug. 5, 2021, which is fully incorporated by reference herein.

### INCORPORATION BY REFERENCE OF SEQUENCE LISTING

The nucleic and/or amino acid sequences provided herewith are shown using standard letter abbreviations for nucleotide bases, and one letter code for amino acids, as defined in with 37 CFR 1.831 through 37 CFR 1.835. Only one strand of each nucleic acid sequence is shown, but the complementary strand is understood as included by any reference to the displayed strand.

The Sequence Listing is submitted as an XML file named 95083_410_3_SEQ created Feb. 10, 2025, about 14000 Bytes, which is incorporated by reference herein in its entirety.

### FIELD

The present invention relates to combination medicaments for use in patients diagnosed with cancer, also referred to as malignant neoplastic disease herein. The combination medicaments according to the invention comprise a soluble HLA (human leukocyte antigen) heavy chain polypeptide and an inhibitor of the interaction between CD47 and signal regulatory protein alpha (SIRPα).

### BACKGROUND OF THE INVENTION

The tumor microenvironment contains a large number of macrophages, which can form up to 50% of the tumor mass. Importantly, macrophages perform immune surveillance. Therefore, both innate immunity and tumor-associated macrophages have gradually attracted attention as a target of innate immune therapy. However, there is a complex relationship between tumors and macrophages, which can actually promote tumor growth or metastasis in some cases.

The dual role of macrophages was shown to originate from their great heterogeneity and plasticity. Macrophages can be broadly divided into two broad groups: type 1 or classically activated (M1), and type 2 or alternatively activated (M2). In vitro, M1 cells are characterized by a proinflammatory phenotype and display microbicidal activity, which results in tumor suppression, whereas M2 macrophages can promote tissue repair, matrix remodeling and angiogenesis supporting tumorigenesis. Different macrophage phenotypes have different phagocytic activities. In the context of cancer, macrophages quickly detect membrane molecules on tumor cells and are able to engulf tumor cells through phagocytosis, a multi-step cellular process involving target cell recognition, cellular engulfment, and lysosomal digestion, regulated by receptor-ligand interactions between the target cell and the phagocyte. Multiple antiphagocytic signals present in cancer cells have been identified, which include LILRB1 and LILRB2. The leukocyte Ig-like receptor subfamily B (LILRB) is a group of type I transmembrane glycoproteins with extracellular Ig-like domains that bind ligands and intracellular ITIMs that can recruit tyrosine phosphatases SHP-1, SHP-2, and the inositol phosphatase SHIP.

Macrophages express both LILRB1 and LILRB2 receptors, and activation of these receptors downmodulates macrophage activity in the tumor microenvironment. LILRB1 blockade in immune cells has demonstrated efficacy in solid and liquid cancer using in vitro models. LILRB1 signaling, for instance, can inhibit monocyte activation and macrophage phagocytosis (Colonna M. et al. 1997 J. Exp. Med. 186 (1): 1809). LILRB2 blockade reprograms TAMs into a proinflammatory phenotype, suppresses T regulatory cell infiltration, and promotes efficacy of immune checkpoint inhibitors. Furthermore, blockade of LILRB2 inhibits receptor-mediated activation of SHP-1/SHP-2 and enhances proinflammatory responses (Alsina-Beauchamp D. et al. 2018, J. Clin. Invest. 128 (12): 5647).

ImmunOs Therapeutics have developed human leukocyte antigen (HLA) heavy chain-based molecules that bind to LILRB1, LILRB2 and KIR3DL1 receptors (see for example, WO 2017153438 A1).

Based on the above-mentioned state of the art, the objective of the present invention is to provide means and methods to enhance the anti-tumor effect of therapeutic HLA heavy chain-based molecules. This objective is attained by the subject-matter of the independent claims of the present specification, with further advantageous embodiments described in the dependent claims, examples, figures and general description of this specification.

### SUMMARY OF THE INVENTION

Interaction of inhibitory CD47 binds to SIRPα to inhibit phagocytosis, and may be targeted by checkpoint inhibitors that bind to either CD47 or SIRPα to improve immune outcomes. HLA class I heavy chain fusion proteins previously developed by the inventors blocking LILRB1/2 immune checkpoint inhibitors share an ability to modulate phagocytosis checkpoints. The inventors designed experiments to assess whether agents which inhibit binding of CD47 to SIRPα can enhance the proinflammatory effect of HLA fusion proteins on macrophage phagocytosis of tumor cells (FIG. 1). The results demonstrate that the HLA fusion protein candidate iosH2 increases the phagocytosis activity of human primary macrophages against solid and liquid cancers, as both monotherapy, and particularly in combination with anti-CD47 and anti-SIRPα antibodies.

A first aspect of the invention relates to a combination medicament, said combination comprising:
1. A soluble MHC class I HLA heavy chain polypeptide, optionally stabilized by fusion to a polypeptide moiety conferring enhanced in-vivo stability; and
2. an inhibitor of the interaction between CD47 and SIRPα.

In particular embodiments, the soluble HLA heavy chain polypeptide is provided as an HLA fusion protein that comprises an HLA heavy chain polypeptide (the extracellular domain of an HLA heavy chain), joined to an immunoglobulin crystallizable fragment (Ig Fc) polypeptide. In more particular embodiments of the combination medicament, the HLA heavy chain polypeptide is selected from HLA-B57, HLA-C08, HLA-A25, HLA-B58, HLA-B27, HLA-A30, HLA-B53, or HLA-C12. In other particular embodiments, the HLA heavy chain polypeptide is a variant at least (2) 95%, similar in sequence to an HLA heavy chain as defined above, with a similar biological activity.

In certain particular embodiments, the HLA fusion protein is associated with a beta-2-microglobulin (β2m) polypeptide.

Another aspect relates to the use of the combination medicament according to the invention in treatment of cancer, particularly solid tumors (malignant neoplastic disease).

Another aspect of the invention relates to an inhibitor of the interaction between CD47 and SIRPα for use in the treatment of cancer, also referred to herein as a malignant neoplastic disease, when administered prior to, in combination with, or subsequent to, a soluble HLA heavy chain polypeptide, optionally stabilized by fusion to a polypeptide moiety conferring enhanced in-vivo stability, particularly an HLA fusion protein, as specified herein.

Yet another aspect of the invention relates a soluble HLA heavy chain polypeptide, optionally stabilized by fusion to a polypeptide moiety conferring enhanced in-vivo stability, for use in treatment of cancer, particularly solid tumors (malignant neoplastic disease), when administered prior to, in combination with, or subsequent to, an inhibitor of the interaction between CD47 and SIRPα as specified herein.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having”, “containing”, and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of an RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

In the context of the present specification, the terms sequence identity, sequence similarity and percentage of sequence identity refer to a single quantitative parameter representing the result of a sequence comparison determined by comparing two aligned polypeptide sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. Unless stated otherwise, sequence identity values provided herein refer to the value obtained using the BLAST suite of programs (Altschul et al., J. Mol. Biol. 215:403-410 (1990)) using the above identified default parameters for protein.

Reference to identical sequences without specification of a percentage value implies 100% identical sequences (i.e. the same sequence).

As used herein, the term pharmaceutical composition refers to a soluble HLA heavy chain, particularly an HLA fusion protein, and or an inhibitor of the interaction between CD47 and SIRPα, together with at least one pharmaceutically acceptable carrier. In certain embodiments, the pharmaceutical composition according to the invention is provided in a form suitable for parenteral, particularly injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease, or at least one of the clinical symptoms thereof, for example, slowing, or reducing tumor growth). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

The terms “cancer” and “malignant neoplastic disease” are used synonymously herein. Particular alternatives of any of the aspects and embodiments disclosed herein are directed at the use of the combinations of the invention in treatment of solid tumours. Other alternatives of any of the aspects and embodiments disclosed herein are directed at the use of the combinations of the invention in treatment of liquid cancers such as myelogenous or granulocytic leukemia, particularly AML, lymphatic, lymphocytic, or lymphoblastic leukemia and lymphoma, polycythemia vera or erythremia.

In the context of the present specification, the term peptide linker, or amino acid linker refers to a polypeptide of variable length that is used to connect two polypeptides in order to generate a single chain polypeptide. Exemplary embodiments of linkers useful for practicing the invention specified herein are oligopeptide chains consisting of 1, 2, 3, 4, 5, 10, 20, 30, 40 or 50 amino acids. A non-limiting example of an amino acid linker is the polypeptide GGGGSGGGGS (SEQ ID NO 003) that links an HLA heavy chain polypeptide with a stabilizing peptide, for example, linking an HLA-B57 with an IgG4 Fc polypeptide in an HLA fusion protein.

The term Human leukocyte antigen (HLA) heavy chain, HLA heavy chain in the context of the present specification relates to the protein encoded by an MHC Class I histocompatibility antigen gene, particularly a classical, MHC class 1a heavy chain. In humans, an HLA heavy chain can be a monomer, or form a part of dimeric structures comprising a heavy chain with three extracellular domains (α1, α2, and α3), bound non-covalently to a 2m light chain, or optionally, trimeric structures wherein a small peptide is associated at the peptide-binding cleft. Full length HLA heavy chain polypeptides comprise an extracellular domain comprising an α1, an α2, and an α3 domain, a transmembrane domain, and an intracellular domain.

A list of naturally occurring HLA heavy chains which are considered embodiments of the term according to this aspect of the invention are listed in Table 1.

TABLE 1
List of HLA heavy chain alleles

| | | |
|---|---|---|
| HLA-A | HLA-B | HLA-C |

| | | | |
|---|---|---|---|
| A01 | B07 | B53 | C01 |
| A02 | B08 | B54 | C02 |
| A03 | B13 | B55 | C03 |
| A11 | B14 | B56 | C04 |
| A23 | B15 | B57 | C05 |
| A24 | B18 | B58 | C06 |
| A25 | B27 | B59 | C07 |
| A26 | B35 | B67 | C08 |
| A29 | B37 | B73 | C12 |
| A30 | B38 | B78 | C14 |
| A31 | B39 | B81 | C15 |
| A32 | B40 | B82 | C16 |
| A33 | B42 | B83 | C17 |
| A34 | B44 | | C18 |
| A36 | B46 | | |
| A43 | B47 | | |
| A66 | B48 | | |
| A68 | B49 | | |
| A69 | B50 | | |
| A74 | B51 | | |
| A80 | B52 | | |

The term variant in the context of the present specification relates an HLA heavy chain polypeptide sequence with at least one amino acid residue that differ from a naturally-occurring polypeptide sequence. For example, a variant HLA heavy chain polypeptide in which one, or several amino acid substitutions have been introduced, such that it differs from the original, naturally occurring HLA heavy chain polypeptide sequence it is derived from. A variant HLA heavy chain polypeptide is characterized by a sequence similarity of at least (2) 95%, particularly >98% compared to the aligned naturally occurring extracellular domain of an HLA heavy chain from which it is derived. In addition, the altered amino acids do not impede the capacity of the variant HLA heavy chain to interact with its ligands, such that the variant has a similar biological activity to the original sequence from which it is derived.

The biological activity of a variant HLA heavy chain peptide may be assessed in the context of incorporation into an HLA fusion protein according to the invention, specifically by measuring the capacity for binding to the ligand LILRB2. A similar biological activity of a variant is defined as at least 65%, particularly 85%, or even 95% of the capacity of a variant HLA heavy chain polypeptide used in an HLA fusion protein to bind to LILRB2, compared to the equivalent non-variant sequence, as measured by an enzyme-linked immunosorbent assay (ELISA) method. This may be assessed by calculating the EC50, i.e. the concentration of a fusion protein that gives a half-maximal response, in this case, half the maximal binding to a biotinylated LILRB2 molecule. For example, for the variant HLA-B57 heavy chain-IgG4 fusion protein assessed in the examples (FIG. 3), the equivalent non-variant wildtype HLA-B57-based structure has an EC50 of LILRB2 binding of approximately 21 nM (nanomole/L). The threshold for EC50 for a suitable variant as measured by ELISA with approximately 65% biological function is therefore approximately 32 nM, 85% is approximately 29 nM, and 95% is approximately 22 nM.

To determine the EC50 of LILRB2 binding according to the invention, streptavidin coated high binding capacity 96 well plates are coated with 50 μl of c-terminally biotinylated LILRB2 (for example, obtained from BPS Bioscience #100335) at a final concentration of 5 μg/ml in PBS buffer. PBS and IgG isotype may be used as negative controls. A serial dilution of HLA fusion protein is applied in a titrated series, (for example, eight concentration points: 10, 2.5, 1, 0.25, 0.1, 0.025, 0.01, 0.0025 μg/ml), preferably applied in 50 μl duplicates. A labelled antibody that can detect the fusion protein (for example, if the fusion protein comprises an IgG Fc, an APC conjugated goat anti-human IgG antibody (Jackson Immuno Research #109-135-098, 1:100 dilution in TBS 50 μl) may then be applied to detect HLA fusion protein binding. Finally, 50 μl TBS is added to each well, and fluorescence excitation and emission measured at the appropriate wavelength (for example 650 nm & 660 nm, respectively). A three-parameter based log (agonist) model is one suitable means to determine the EC50 of the HLA fusion protein binding to the LILRB2.

The term extracellular domain as applied to an HLA heavy chain or a variant of an HLA heavy chain in the context of this specification, refers to the extracellular portion of an HLA heavy chain protein (or a variant protein in which amino acid substitutions have been introduced into a naturally occurring HLA heavy chain protein sequence). The extracellular portion of an HLA Class 1a polypeptide comprises the alpha (α) 1 domain, and α2 domain, and an α3 domain, which are essential for receptor ligand interactions which mediate the immunomodulatory effects of the HLA fusion protein in the pharmaceutical composition for use according to the invention. The extracellular domain excludes the transmembrane domain, and the intracellular domain.

In the context of the present specification, the term HLA fusion protein refers to a recombinant polypeptide which comprises of the extracellular domain of an HLA heavy chain, joined to a stabilizing domain, particularly a stabilizing immunoglobulin (Ig) Fc, optionally by means of a peptide linker. Particular fusion proteins of use in the context of the present invention are disclosed in PCT/EP2016/052317, published as WO2016 124661 A1; PCT/EP2017/055373, published as WO2017 153438 A1; and PCT/EP2017/070255, published as WO2018 029284 A1, all of which are incorporated herein by reference. The term HLA fusion protein encompasses an HLA fusion protein in complex with a 2-microglobulin polypeptide as secreted from mammalian cell culture, as well as purified HLA fusion protein which is not associated with 82-microglobulin. The term HLA fusion protein may refer to a monomer comprising a single HLA polypeptide joined to a single stabilizing immunoglobulin (Ig) Fc domain, or a dimer formed by association of a first HLA fusion protein monomer, and a second HLA fusion protein monomer, particularly joined via stabilizing Ig Fc domains.

In the context of the present specification, the term 2-microglobulin (62m, B2m, B2M), B2m polypeptide, or 2m polypeptide refers to the beta (B) chain, also known as the HLA light chain, of MHC class I heterodimers. The term 82-microglobulin encompasses firstly a pre-processing 32-microglobulin comprising a secretory signal, for example, the sequence of Uniprot P61769, or the sequence SEQ ID NO 006, and particularly the post-secretion form of the protein, in which a secretory signal portion of the protein has been removed by cleavage during the secretion process . . .

In the context of the present specification, the term secretory signal, secretory signal peptide or signal sequence refers to an N-terminal leader sequence initiating the open reading frame (ORF) of a polypeptide, usually about 6-30 amino acids in length. In rare cases, a secretory signal is placed at the C-terminus of a polypeptide. Secretory signals are sometime referred to as targeting signals, localization signals, transit peptides, leader sequences, or leader peptides. Secretory signals which enable efficient secretion of a polypeptide from cells are well known in the art, and may be included in the ORF of a recombinant protein in order to facilitate export of a polypeptide to the supernatant in cell-based polypeptide manufacturing system, allowing purification of a polypeptide from the cell supernatant. Upon translation of the mRNA encoding the secretory signal, it is recognized by a cytosolic protein mediating transfer of the mRNA-ribosome complex to a channel protein in the endoplasmic reticulum (ER). The newly synthesized polypeptide comprising the secretory signal peptide is translocated to the ER lumen through the channel protein, entering the cell secretion pathway. Signal sequences of particular use according to the invention are those that are cleaved from the final polypeptide product following translation, for example, SEQ ID NO 004.

### Binding; Binders Ligands Antibodies:

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷ mol/L when binding to its target, but a dissociation constant at least three orders of magnitude higher in its interaction with a molecule having a globally similar chemical composition as the target, but a different three-dimensional structure.

In the context of the present specification, the term dissociation constant (K_{D}) is used in its meaning known in the art of chemistry and physics; it refers to an equilibrium constant that measures the propensity of a complex composed of [mostly two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ A b ] * [ A g ]}{[ A b A g ]}$$

[Ab]: concentration of antibody; [Ag]: concentration of antigen; [AbAg]: concentration of antibody-antigen complex

In the context of the present specification, the terms off-rate (K_{off}; [1/sec]) and on-rate (Kₒₙ; [L/(sec*mol)]) are used in their meaning known in the art of chemistry and physics; they refer to a rate constant that measures the dissociation (K_{off}) or association (Kₒₙ) of 5 an antibody with its target antigen. K_{off} and Kₒₙ can be experimentally determined using methods well established in the art. A method for determining the K_{off} and Kₒₙ of an antibody employs surface plasmon resonance. This is the principle behind biosensor systems such as the Biacore® or the ProteOn® system. They can also be used to determine the dissociation constant K_{D} by using the following formula:

$${K}_{D} = \frac{[ {K}_{o f f} ]}{[ {K}_{o n} ]}$$

The natural upper limit for the on-rate Kₒₙ is 109 L/(sec*mol).

In the context of the present specification, the term antibody refers to whole antibodies including but not limited to immunoglobulin type G (IgG), type A (IgA), type D (IgD), type E (IgE) or type M (IgM), any antigen-binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region of IgG is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

The term antibody-like molecule in the context of the present specification refers to a molecule capable of specific binding to another molecule or target with high affinity/a K_{d}≤10E⁻⁸ mol/l. An antibody-like molecule binds to its target similarly to the specific binding of an antibody. The term antibody-like molecule encompasses a repeat protein, such as a designed ankyrin repeat protein (Molecular Partners, Zürich), an engineered antibody mimetic protein exhibiting highly specific and high-affinity target protein binding (see US2012142611, US2016250341, US2016075767 and US2015368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins. The term antibody-like molecule further encompasses a specifically binding polypeptide derived from a protein A domain, a fibronectin domain FN3, a consensus fibronectin domain, a lipocalin (see Skerra, Biochim. Biophys. Acta 2000, 1482 (1-2): 337-50), a polypeptide derived from a Zinc finger protein (see Kwan et al. Structure 2003, 11 (7): 803-813), a Src homology domain 2 (SH2) or Src homology domain 3 (SH3), a PDZ domain, a gamma-crystallin, ubiquitin, a cysteine knot polypeptide or a knottin, cystatin, Sac7d, a triple helix coiled coil (also known as alphabodies), a Kunitz domain or a Kunitz-type protease inhibitor and a carbohydrate binding module 32-2.

The term protein A domains derived polypeptide refers to a molecule that is a derivative of protein A and is capable of specifically binding the Fc region and the Fab region of immunoglobulins.

The term armadillo repeat protein refers to a polypeptide comprising at least one armadillo repeat, wherein an armadillo repeat is characterized by a pair of alpha helices that form a hairpin structure.

The term humanized camelid antibody in the context of the present specification refers to an antibody consisting of only the heavy chain or the variable domain of the heavy chain (VHH domain) and whose amino acid sequence has been modified to increase their similarity to antibodies naturally produced in humans and, thus show a reduced immunogenicity when administered to a human being. A general strategy to humanize camelid antibodies is shown in Vincke et al. “General strategy to humanize a camelid single-domain antibody and identification of a universal humanized nanobody scaffold”, J Biol Chem. 2009 Jan. 30; 284 (5): 3273-3284, and US2011165621A1.

In the context of the present specification, the term immunoglobulin crystallizable fragment (Fc) region, or Ig Fc refers to a fraction of an antibody, or immunoglobulin (Ig), consisting of a C_{H}2 and a C_{H}3 domain. Ig Fc encompasses both a monomer, or a dimer comprising two Ig Fc, covalently linked by disulfide bonds. In the context of the HLA fusion protein according to the invention, disulfide bonds can join two HLA fusion proteins molecules, each comprising Ig Fc domains. The presence of the Ig Fc in the HLA fusion protein facilitates increased solubility, stability, avidity, half-life, and from a technological point of view, cost-effective production and purification in mammalian systems (protein A or G purification).

In the context of the present specification, the term inhibitor of the interaction between CD47 and SIRPα encompasses any agent capable of disrupting an inhibitory signaling cascade between CD47 and SIRPα that limits phagocyte activation. This includes antibodies, antibody fragments and antibody-like molecules capable of specific binding to either CD47 or SIRPα. It also includes or soluble recombinant proteins which incorporate the extracellular ligand-binding domains of either CD47, or SIRPα.

The term signal-regulatory protein alpha (SIRPα, Uniprot P78324) refers to the immunoglobulin-like surface receptor for CD47 (Uniprot Q08722). Ligation of CD47 and SIRPα mediates negative regulation of phagocytosis.

### DETAILED DESCRIPTION OF THE INVENTION

A first aspect of the invention relates to a combination medicament, particularly for use in treating a cancer. The combination medicament comprises:
1. A soluble HLA heavy chain molecule, optionally stabilized as an HLA fusion protein, and
2. an inhibitor of the interaction between CD47 and SIRPα.

In some embodiments, these two agents are present in a single pharmaceutical composition. In other embodiments, the soluble HLA heavy chain molecule (particularly as provided as an HLA fusion protein) is provided for use in a patient who is receiving a medicament comprising said an inhibitor of the interaction between CD47 and SIRPα. In other words, an HLA fusion protein is provided for use in a cancer patient who is scheduled receive an inhibitor of the interaction between CD47 and SIRPα in the following weeks, or month, or has recently received an inhibitor of the interaction between CD47 and SIRPα in the weeks or month prior. Alternatively, the two agents may be administered in an overlapping dosage regime.

Any mention herein of a combination medicament according to the invention encompasses the re-formulation of a specific claim, item, aspect or embodiment having the same limitations, directed at an HLA heavy chain or HLA fusion protein for use as specified, administered in combination with an inhibitor of the interaction between CD47 and SIRPα.

### HLA Fusion Proteins

The HLA fusion protein component of the combination medicament for use according to the invention, or the inhibitor of the interaction between CD47 and SIRPα for use according the invention, comprises an HLA heavy chain polypeptide which is the extracellular domain of an HLA heavy chain. For pharmaceutical use, the HLA heavy chain benefits from stabilization by fusion to a protein moiety that confers enhanced pharmaceutical manufacturability and stability, and enhanced plasma half-life. In particularly advantageous embodiments, the inventors found that joining the HLA heavy chain to an immunoglobulin crystallizable fragment (Ig Fc) polypeptide confers these benefits. The invention is further described in terms of such fusion proteins with IgG Fc domains, but the skilled person recognizes that other modalities of stabilization will be available to confer similar benefits.

Certain domains of HLA heavy chain proteins not required for cognate ligand interactions, are not necessarily included in the HLA polypeptide portion of the HLA fusion protein comprised in the combination medicament for use according to the invention. In particular embodiments, the intracellular domain, and the transmembrane domain are absent from the HLA heavy chain polypeptide.

In particular embodiments of the methods to produce an HLA fusion protein, or the isolated HLA fusion protein according to the invention, the variant HLA-B57 polypeptide comprised in the HLA fusion protein includes the alpha 1, 2 and 3 domains of the naturally occurring HLA heavy chain protein, regions of which are essential for receptor ligand interactions which mediate the immunomodulatory effects of an HLA fusion protein according to the invention.

In more particular embodiments of the method to produce an HLA fusion protein, or the isolated HLA fusion protein according to the invention, the variant HLA-B57 polypeptide protein is the alpha 1, 2 and 3 domains of the naturally occurring HLA heavy chain protein, excepting the C-terminal isoleucine-valine dipeptide, preceded by the threonine-valine-proline residues of the extracellular domain, within the HLA-B57 region preceding the transmembrane domain sometimes annotated, or referred to, as the “connecting peptide”.

Structural data suggests that HLA heavy chains interact with ligands such as Killer immunoglobulin-like receptors (KIR) and leukocyte immunoglobulin-like receptors (LILR) via regions distant from the transmembrane region. Amino acids close to the membrane do not generally interact with receptors. Furthermore, the inventors surmise that the high content of hydrophobic amino acids within the 5 C-terminal amino of the extracellular domain of naturally occurring HLA heavy chain sequences, is likely to introduce undesirable properties into recombinant proteins, such as a tendency towards protein aggregation, which can then affect the production, purification, stability and toxicity in downstream production processes.

In particular embodiments of the combination medicament, or the inhibitor of the interaction between CD47 and SIRPα for use according the invention, the HLA heavy chain extracellular domain polypeptide of the HLA fusion protein component comprises the core structure of the extracellular portion of an HLA heavy chain protein sequence, comprising the alpha 1, 2, and 3 domains, as this portion confers the HLA fusion protein with the ability to interact with surface molecules on target cells.

In certain embodiments of the combination medicament, or the inhibitor of the interaction between CD47 and SIRPα for use according the invention, the HLA heavy chain polypeptide portion of the HLA fusion protein has the polypeptide sequence of the extracellular domain of a naturally occurring HLA heavy chain listed in Table. 1. In particular embodiments, the HLA heavy chain extracellular domain is that of a naturally occurring HLA heavy chain with immunomodulatory qualities, such as specific binding to regulatory KIR3DL1, LILRA and LILRB1/2 cell surface proteins which alter innate and adaptive immune cell function.

In certain embodiments, the HLA heavy chain portion of the HLA fusion protein is derived from the extracellular domain of HLA-B58. In particular embodiments, the combination medicament comprises a soluble HLA heavy chain polypeptide which is an HLA-B58 fusion protein polypeptide with the sequence designated SEQ ID NO 010.

In certain embodiments, the HLA heavy chain portion of the HLA fusion protein is derived from the extracellular domain of HLA-A30. In particular embodiments, the combination medicament comprises a soluble HLA heavy chain polypeptide which is an HLA-B58 fusion protein polypeptide with the sequence designated SEQ ID NO 011.

In certain embodiments, the HLA heavy chain portion of the HLA fusion protein is derived from, or essentially is, the extracellular domain of HLA-B27.

In certain embodiments, the HLA heavy chain portion of the HLA fusion protein is derived from, or essentially is, the extracellular domain of HLA-B44.

In certain embodiments, the HLA heavy chain portion of the HLA fusion protein is derived from, or essentially is, the extracellular domain of HLA-B81.

In certain embodiments, the HLA heavy chain portion of the HLA fusion protein is derived from, or essentially is, the extracellular domain of HLA-C08. In particular embodiments, the combination medicament comprises a soluble HLA heavy chain polypeptide which is an HLA-C08 fusion protein polypeptide with the sequence designated SEQ ID NO 009.

In further embodiments, the HLA heavy chain portion of the HLA fusion protein is derived from, or essentially is, the extracellular domain of HLA-C12.

In particular embodiments, the HLA heavy chain portion of the HLA fusion protein is derived from, or essentially is, the extracellular domain of HLA-B57.

In further embodiments of the combination medicament, or the inhibitor of the interaction between CD47 and SIRPα for use according the invention, the HLA fusion protein comprises a variant HLA heavy chain extracellular domain polypeptide. Said variant HLA heavy chain polypeptide is characterized by a sequence similarity (on the protein level) of at least (≥) 95% to the non-variant extracellular domain of an HLA heavy chain, and has a similar biological activity (as defined under the term similar biological activity). In particular embodiments, the variant HLA heavy chain is ≥98% similar to the naturally occurring HLA heavy chain extracellular domain from which it is derived.

In particular embodiments of the combination medicament, or the inhibitor of the interaction between CD47 and SIRPα for use according the invention, the HLA fusion protein is associated with a beta-2-microglobulin (β2m) polypeptide. Using the β2m-associated HLA fusion protein confers the advantage of increased yields of the immunomodulatory HLA fusion protein, as producing this product does not incur the loss of protein that occurs during a β2m disassociation step (FIG. 2). In some embodiments of the combination medicament, or the inhibitor of the interaction between CD47 and SIRPα for use according the invention, the a β2m polypeptide is linked by a peptide linker to the HLA fusion protein. In particular embodiments of the combination medicament according to the invention, the β2m polypeptide is non-covalently associated with said HLA fusion protein. In particular embodiments, the HLA fusion protein is associated with a β2m molecule at a molar ratio of between 3:5 to 7:5. In more particular embodiments, the ratio is between 4:5 to 6:5. In other words, the HLA fusion protein and the β2m polypeptide are present at a ratio of, or close to, 1 to 1.

In particular embodiments of the combination medicament, or the inhibitor of the interaction between CD47 and SIRPα for use according the invention, HLA fusion protein comprises a variant HLA-B57 heavy chain extracellular domain polypeptide, which is characterized by at least one, or two amino acid substitutions which differ from the polypeptide sequence a naturally-occurring MHC class 1a heavy chain extracellular domain polypeptide. The HLA-B57 heavy chain gene family currently encompasses 221 known variants with unique nucleic acid sequences, numbering HLA-B*57:01 to HLA-B*57:141, encoding several dozen unique protein sequences. The protein sequence for which are known, and may be retrieved, for example, by entering the search term “B*57” into the MGT/HLA Allele Query Form provided by the European Bioinformatics Institute Immuno Polymorphism Database, Robinson J. et al. 2013 Nucleic Acids Res. 41: D1234, https://www.ebi.ac.uk/ipd/imgt/hla/allele.html). In more particular embodiments, the HLA heavy chain polypeptide is a variant of the naturally occurring extracellular domain of an HLA-B57 heavy-chain polypeptide sequence, in which one, or two amino acid substitutions have been performed such that it is characterized by an E at position 46, and an R at position 97 (numbering assigned from the G, S, H motif indicating the start of the extracellular domain). In other words, an amino acid other than E has been replaced with an E at position 46, and/or an amino acid other than R, has been replaced with an R at position 97. The numbering of these amino acids refers to the assignment of integers sequentially beginning with the G, S, H motif that initiates the extracellular domain of a secreted HLA-B57 portion, lacking the secretion signal, with the numbers 1, 2, and 3. The inventors find these substitutions correlate with high yields of the resulting variant-based HLA fusion protein compared to the wildtype sequence (FIG. 2).

In particular embodiments of the combination medicament, or the inhibitor of the interaction between CD47 and SIRPα for use according the invention, the β2m polypeptide associated with the HLA fusion protein has the sequence designated SEQ ID NO 006.

In more particular embodiments of the combination medicament, or the inhibitor of the interaction between CD47 and SIRPα for use according the invention, the HLA heavy chain polypeptide portion of the HLA fusion protein comprises the variant HLA-B57 sequence designated SEQ ID NO 001. In still more particular embodiments, the HLA heavy chain polypeptide essentially consists of the sequence designated SEQ ID NO 001.

In further particular embodiments of combination medicament, or the inhibitor of the interaction between CD47 and SIRPα for use according the invention, the HLA heavy chain polypeptide and the Ig Fc polypeptide of the HLA fusion protein are joined by a peptide linker. In particular embodiments, the peptide linker is between 5 and 20 amino acids in length. In more particular embodiments this joining peptide linker has the sequence SEQ ID NO 003.

In particular embodiments of the combination medicament, or the inhibitor of the interaction between CD47 and SIRPα for use according the invention, the HLA fusion protein comprises a polypeptide with the sequence designated SEQ ID NO 005. In other embodiments, the HLA fusion protein additionally comprises a secretory signal upstream of the sequence SEQ ID NO 005. In particular embodiments, the secretory signal upstream of the HLA fusion protein polypeptide is SEQ ID NO 004. In other embodiments, the HLA fusion protein essentially consists of a secretory signal of SEQ ID NO 004, joined to a polypeptide designated SEQ ID NO 005.

In more particular embodiments of the combination medicament, or the inhibitor of the interaction between CD47 and SIRPα for use according the invention, the HLA fusion protein essentially consists of the sequence designated SEQ ID NO 005 (comprising a variant extracellular domain of HLA-B57 fused to an IgG4 Fc), associated with a β2m polypeptide.

Natural HLA molecules expressed in the endoplasmic reticulum of human cells associate with peptide epitopes before undergoing transport and display on the cell surface. Binding of peptide to HLA class molecules is thought to change their conformation, which may affect interactions with binding partners such as LILRB1 and LILRB2. The binding affinity and immunomodulatory effects of an HLA-B57 polypeptide associated with β2m polypeptide, not further associated with a peptide epitope are demonstrated in FIG. 4 of the Examples. In particular embodiments of the pharmaceutical composition according to the invention, the HLA polypeptide is not associated with a peptide epitope. In other words, the antigen-binding groove, or antigen-binding cleft formed by the alpha 1 and alpha 2 domains of the HLA polypeptide is not bound to a small, antigenic peptide.

### HLA Fusion Protein Stabilizing and Linking Peptides

The HLA fusion portion of the combination medicament, or the inhibitor of the interaction between CD47 and SIRPα for use according the invention, further comprises a polypeptide conferring stability during expression and purification. The presence of stabilizing portion of the HLA fusion protein increases the yield and solubility by reducing degradation and oligomerization of the HLA fusion protein, as well as increasing viability of the cell expressing the fusion protein.

In particular embodiments of the combination medicament, or the inhibitor of the interaction between CD47 and SIRPα for use according the invention, the stabilizing polypeptide of the HLA fusion protein is a human Ig Fc polypeptide. An Ig Fc portion may also prolong the in vivo half-life of a molecule in vivo by binding to recycling receptors.

In particular embodiments of the combination medicament, or the inhibitor of the interaction between CD47 and SIRPα for use according the invention, the stabilizing polypeptide is an isotype IgG Fc. An IgG Fc stabilizing peptide domain delivers an added advantage during purification of the HLA fusion protein, by enabling absorption to a protein A or G coated surface. The inventors consider a possible alternative that may provide similar benefits if attached to the functional HLA polypeptides in lieu of Ig Fc could be bovine serum albumin. Previous work by the inventors has established that an albumin molecule, such as bovine serum albumin, may also serve as a stabilizing polypeptide. It is also known that PEGylation can enhance the half-life of proteins in circulation, hence it is another feasible stabilizing peptide.

In particular embodiments of the combination medicament, or the inhibitor of the interaction between CD47 and SIRPα for use according the invention, the HLA fusion protein comprises an IgG4 polypeptide, which is desirable isotype in therapeutic fusion proteins due to its low cytotoxicity. In still more particular embodiments of the combination medicament for use, or the inhibitor of the interaction between CD47 and SIRPα for use according the invention, the HLA fusion protein comprises an altered IgG4 S228P.dk molecule with sequence SEQ ID NO 002. This is characterized by a mutation in the hinge region of the IgG4, where Proline (P) is substituted for serine(S) at position 228 of the original IgG4 antibody, and dK indicates a deletion of the last amino acid Lysine (K) on the original IgG4 sequence. These changes give the IgG4 format stability and less heterogenicity. Both changes are well established and used commonly in diverse Fc constructs.

In certain embodiments of the combination medicament, or the inhibitor of the interaction between CD47 and SIRPα for use according the invention, the HLA fusion protein comprises an HLA polypeptide joined with an IgG polypeptide as part of a single polypeptide chain by a peptide linker, a short sequence of amino acids 5, 10, 15, or 20 residues in length. In particular embodiments, the peptide linker is a non-immunogenic sequence, such as a sequence rich in serine and glycine residues. In more particular embodiments, the peptide linker has the sequence SEQ ID NO 003.

In particular embodiments of the combination medicament, or the inhibitor of the interaction between CD47 and SIRPα for use according the invention, or the inhibitor of the interaction between CD47 and SIRPα for use according the invention, the HLA fusion protein is in the format of a dimer. Said dimer comprises a first monomer and a second monomer, and each monomer independently of the other monomer comprises an HLA fusion protein, an HLA heavy chain extracellular domain polypeptide fused to an Ig Fc portion, the latter of which may associate via disulfide bonds. Each monomer according to the invention may be additionally associated with a β2m polypeptide. In particular embodiments, neither of the HLA class I components is associated with a peptide epitope.

### Inhibitors of the Interaction Between CD47 and SIRPα.

Inhibitors of the interaction between CD47 and SIRPα described in preclinical and clinical trials encompass various agents capable of disrupting an inhibitory signaling cascade between CD47 and SIRPα that limit phagocyte activation (Zhang W. et al. 2020, Front. Immunol. 11 (19) doi: 10.3389/fimmu.2020.00018). This includes antibodies, antibody fragments, antibody-like molecules or natural ligand receptors capable of binding to either CD47 or SIRPα abrogating their interaction with ligand, or recombinant proteins which incorporate the extracellular ligand-binding domains of either CD47 or SIRPα.

Inhibitors of the interaction between CD47 and SIRPα known in the art include ALX148 (ALX oncology), TTI-662 or TTI-621 (Trillium Therapeutics), Hu5F9-G4 (Stanford University 47), TI-061 (Arch Oncology), SRF231 (Surface Oncology), SHR-1603 (Hengrui), NI-1701, NI-1801 (Novimmune TG Therapeutics), IBI188 (Innovent Biologics), CC-90002 (Celgene Inibrx), AO-176 (Arch Oncology), lemzoparlimap/TJC4 (I-MAB Biopharma), SY102 (Salyuan), SL-172154 (Shattuck Labs), PSTx-23 (Paradigm Shift Therapeutics), PDL1/CD47BsAb (Hanmi Pharmaceuticals), MBT-001 (Morphiex), LYN00301 (Lynkcell), IMM2504, IMM2502, IMM03 (ImmuneOnco Biopharma), and IMC-002 (ImmuneOncia Therapeutics).

In particular embodiments of the combination medicament, or the inhibitor of the interaction between CD47 and SIRPα for use according the invention, said inhibitor of the interaction between CD47 and SIRPα binds to the extracellular domain of CD47 or SIRPα with a dissociation constant (Kd) of at least 10⁻⁷ M. In further particular embodiments, the Kd of this interaction is at least 10⁻⁸ M, or even 10⁻⁹ M.

In some particular embodiments of the combination medicament for use according to the invention, or the inhibitor of the interaction between CD47 and SIRPα for use according the invention, said inhibitor of the interaction between CD47 and SIRPα comprises an antibody, an antibody fragment, or an antibody-like molecule. In other particular embodiments, said inhibitor of the interaction between CD47 and SIRPα essentially is, an antibody, an antibody fragment, or an antibody-like molecule.

In particular embodiments of the combination medicament according to the invention, or the inhibitor of the interaction between CD47 and SIRPα for use according the invention, said inhibitor of the interaction between CD47 and SIRPα comprises an antibody. In particular embodiments, the inhibitor of the interaction between CD47 and SIRPα is an antibody characterized by specific binding to CD47. In more particular embodiments, the inhibitor of the interaction between CD47 and SIRPα is an antibody characterized by specific binding to CD47 disclosed in WO2020/068752 A1, particularly CC-90002 (Celgene). In certain particular embodiments, the inhibitor of the interaction between CD47 and SIRPα is an antibody characterized by specific binding to SIRPα.

The antibody fragment may be a Fab domain or a variable fragment (Fv) domain of an antibody, or a single-chain antibody fragment, which is a fusion protein consisting of the variable regions of light and heavy chains of an antibody connected by a peptide linker. The antibody may also be a single domain antibody, consisting of an isolated variable domain from a heavy or light chain. Additionally, an antibody may also be a heavy-chain antibody consisting of only heavy chains such as antibodies found in camelids. An antibody-like molecule may be a repeat protein, such as a designed ankyrin repeat protein (Molecular Partners, Zürich).

All fragments must retain antigen binding portions characterized by specific binding to either CD47, or SIRPα to function according to the invention. In other particular, the inhibitory agent is an antibody fragment, comprising an antigen binding portion characterized by specific binding to CD47 In other particular embodiments, the inhibitory agent is an antibody fragment, comprising an antigen binding portion characterized by specific binding to SIRPα. In other particular embodiments of the combination medicament according to the invention, or the inhibitor of the interaction between CD47 and SIRPα for use according the invention, said inhibitor of the interaction between CD47 and SIRPα comprises, or essentially is, a fusion protein, comprising a ligand-binding extracellular domain of CD47 or SIRPα, or a variant of a ligand-binding extracellular domain of CD47 or SIRPα, fused to an Ig Fc. In particular embodiments, the inhibitor fusion protein essentially is a SIRPα polypeptide extracellular domain, and an Ig fc domain. In particular embodiments the inhibitor of the interaction between CD47 and SIRPα is selected from ALX148, TTI-622, or TTI-621. ALX148 is a fusion of a modified SIRPα D1 domain to an inactive human IgG1 Fc which binds to CD47 with a Kd of 1E-9M (Kauder S. et al. 2018, PloS One 13 (8): e0201832). TTI-662 and TTI-621 are the extracellular binding domain of human SIRPα fused to IgG4, or IgG1 Fc respectively (Trillium Therapeutics).

Anti-SIRPα antibodies useful as SIRPα inhibitors further include, for example, KWAR23 (Ring et al., 2017, Proc Natl Acad Sci 114 (49): E10578-E10585), CC-95251 (Celgene), BI 765063 (also known as OSE-172; OSE Immunotherapeutics). Further examples of anti-SIRPα antibodies are described in WO 2019/023347, WO 2013/056352, and WO 2018/190719.

Other such antibodies include, but are not limited to, GS-0189 (Gilead, formerly FSI-189), ES-004, ADU1805 (Voets et al., J Immunother Cancer. 2019 Dec. 4; 7 (1): 340).

In certain embodiments, the SIRPα inhibitor is a soluble CD47 polypeptide, e.g., as described in US 2010/0239579. In certain embodiments, a soluble CD47 polypeptide comprises the extracellular domain of CD47, including the signal peptide (SEQ ID NO:2 of WO 2016/118754), such that the extracellular portion of CD47 is typically 142 amino acids in length, and has the amino acid sequence set forth in SEQ ID NO:3 of WO 2016/118754. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the SIRPα Ig fusion protein ALX148.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the SIRPα Ig fusion protein TTI-662.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the SIRPα Ig fusion protein TTI-621.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody Hu5F9-G4.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody TI-061.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody IBI188.

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody CC-90002 (Inhibrx).

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody AO-176 (Arch Oncology).

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody lemzoparlimap/TJC4 (I-MAB Biopharma).

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the bispecific anti-CD47 antibody SL-172154 (Shattuck Labs).

In particular embodiments of the combination medicament according to the invention, the combination comprises firstly, an HLA B57 class I fusion protein consisting of a dimer two polypeptides of SEQ ID NO 005, wherein each HLA heavy chain portion is characterized by association with a β2m polypeptide, and lacks association with a peptide epitope, and secondly, the anti-CD47 antibody IMC-002 (ImmuneOncia Therapeutics).

In some embodiments, the inhibitor of the interaction between CD47 and SIRPα used in the present invention are able, when bound to CD47 or SIRPα, to sterically block interaction with its binding partners. In other embodiments, this interaction is a non-agonist interaction.

### Dosing and Administration of HLA Fusion Protein Pharmaceutical Compositions

Further aspects of the invention relate to a pharmaceutical composition, particularly for use in treating a form of cancer or malignant neoplastic disease, said composition comprising an HLA fusion protein as specified in the section HLA fusion protein, for use in a patient receiving treatment with an inhibitor of the interaction between CD47 and SIRPα.

The pharmaceutical composition, particularly for use in treating cancer, according to the present invention comprises an HLA fusion protein associated with β2m polypeptide, and is typically formulated into pharmaceutical dosage forms to provide an easily controllable dosage of the drug and to give the patient an elegant and easily handled product. Said pharmaceutical composition further contains a pharmaceutically acceptable carrier. In further embodiments, the composition comprises at least two pharmaceutically acceptable carriers.

The dosage regimen for the pharmaceutical composition of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the pharmaceutical composition of the invention may be administered in a single daily dose, or the total daily dosage may be administered in divided doses of two, three, or four times daily. In particular embodiments, the combination medicament is administered every 1, 2 or 3 weeks.

Many procedures and methods for preparing pharmaceutical compositions are known in the art, see for example L. Lachman et al. The Theory and Practice of Industrial Pharmacy, 4th Ed, 2013 (ISBN 8123922892).

Administration in combination, encompasses both simultaneous administration of the inhibitor of the interaction between CD47 and SIRPα and the pharmaceutical composition comprising an HLA fusion protein, or in separate formulations, or administration of one substance immediately prior to, for example, in the week prior to, or at least in the month prior to, or immediately subsequent to, for example, in the week, or at most the month subsequent to, administration of a second substance.

Another aspect of the invention, is an inhibitor of the interaction between CD47 and SIRPα for use in the treatment of cancer, wherein the inhibitor of the interaction between CD47 and SIRPα is administered prior to, in combination with, or subsequent to, an HLA fusion protein.

### Distinct Dosage Forms

In certain embodiments, the combination therapy comprises two distinct dosage forms, for example, wherein said pharmaceutical composition comprising an HLA fusion protein is provided as a dosage form for intra-tumoral delivery, or local delivery in the vicinity of the tumor, for example, by subcutaneous injection, or intra-tumoral injection into a solid tumor, and said inhibitor of the interaction between CD47 and SIRPα is provided as a dosage form for systemic delivery, particularly by intravenous injection. However, the two agents may also be delivered in two similar dosage forms.

In particular embodiments of the combination medicament according to the invention, or the inhibitor of the interaction between CD47 and SIRPα for use according the invention, said inhibitor of the interaction between CD47 and SIRPα is provided in a dosage form suitable for systemic delivery.

In particular embodiments of the combination medicament according to the invention, or the inhibitor of the interaction between CD47 and SIRPα for use according the invention, said HLA fusion protein is provided in a dosage form suitable for systemic delivery.

### One Common Dosage Form

The invention also encompasses the administration of a soluble HLA heavy chain polypeptide, optionally stabilized by fusion to a polypeptide moiety conferring enhanced in-vivo stability, and an inhibitor of the interaction between CD47 and SIRPα as a single dosage form.

### Medical Treatment and Method of Manufacture

The combination medicament, or the inhibitor of the interaction between CD47 and SIRPα according to the invention is provided for use in treating various forms of cancer. Pre-clinical studies on other forms of a similar LILBR2-directed antineoplastic therapy have demonstrated efficacy in renal cancer, and ovarian cancer. The safety and tolerability of LILRB2-targeting antibody MK-4830 is currently under investigation in a clinical trial targeting a wide range of solid organ cancers (mesothelioma, triple negative breast cancer, ovarian cancer, lung cancer, glioblastoma, pancreatic cancer, gastric cancer), in combination with chemotherapy (ClinicalTrials.gov Identifier: NCT03564691). Each of these indication may feasibly be treated with the LILRB2-bining HLA fusion protein combination medicament according to the invention.

In particular embodiments of the combination medicament, or the inhibitor of the interaction between CD47 and SIRPα according to the invention, it is provided for use to treat a blood cancer. The inventors consider the characteristic T cell-exhaustion, and accessibility of circulating blood cancer cells to the T cell, and macrophage activation induced by pharmaceutical compositions according to the invention mean this combination is likely to be effective. In particular such embodiments, the combination medicament is provided for use in a patient diagnosed with multiple myeloma, as modelled by the RPMI-8226 cell line (FIG. 4).

In other particular embodiments, combination medicament, or the inhibitor of the interaction between CD47 and SIRPα for use according to the invention, is provided for use in a patient diagnosed with a solid cancer, or a metastasis of a solid cancer. In other particular embodiments, the combination is provided for use in a patient diagnosed with a form of breast cancer. In more particular embodiments, the cancer is estrogen receptor positive. In other particular embodiments, the cancer is progesterone receptor positive. In other particular embodiments, the cancer is human epidermal growth factor receptor 2 positive.

Similarly, within the scope of the present invention is a method of treating a cancer patient, comprising administering to the patient the combination medicament, or the inhibitor of the interaction between CD47 and SIRPα according to the invention. The invention further encompasses, as an additional alternative aspect, the use of the components of a combination medicament as specified in detail above, for use in a method of manufacture of a medicament for the treatment or prevention of cancer.

Dosage forms may be for parenteral administration such as subcutaneous, intravenous, intrahepatic or intramuscular injection forms. Optionally, a pharmaceutically acceptable carrier and/or excipient may be present.

### The Invention Further Encompasses the Following Items:

Item 1. A combination medicament for use in treating a malignant neoplastic disease comprising:
   a. a human leukocyte antigen (HLA) fusion protein comprising
      an HLA heavy chain polypeptide selected from:
         an extracellular domain of an HLA heavy chain, particularly an HLA heavy chain selected from HLA-B57, HLA-C08, HLA-A25, HLA-B58, HLA-B27, HLA-A30, HLA-B53, or HLA-C12; or
         a variant of said extracellular domain of an HLA heavy chain, wherein said variant is characterized by a sequence similarity of at least (≥) 95%, particularly ≥98%, and a similar biological activity; and
      an immunoglobulin crystallizable fragment (Ig Fc) polypeptide, particularly an IgG Fc polypeptide, more particularly an IgG4 Fc polypeptide; and
   a. an inhibitor of the interaction between CD47 and alpha (SIRPα).
Item 2. The combination medicament for use according to item 1, wherein the HLA fusion protein is associated with a β2m polypeptide.
Item 3. The combination medicament for use according to item 1 or 2, wherein the HLA fusion protein is non-covalently associated with the β2m polypeptide at a ratio of between 3:5 to 7:5, more particularly between 4:5 to 6:5, more particularly at a ratio of 1:1.
Item 4. The combination medicament for use according to item 3, wherein the HLA heavy chain polypeptide is a variant of the extracellular domain of HLA-B57, and wherein HLA heavy chain polypeptide is characterized by an E at position 46, and an R at position 97.
Item 5. The combination medicament for use according to any one of the items 1 to 4, wherein the HLA heavy chain polypeptide comprises, or essentially consists of, the sequence SEQ ID NO 001.
Item 6. The combination medicament for use according to any one of the items 1 or 5, wherein the HLA fusion protein comprises:
   a. the HLA heavy chain polypeptide as specified in any one of the items 1 to 5;
   b. an IgG Fc polypeptide, particularly an IgG4 F polypeptide, more particularly an IgG4 Fc polypeptide with the sequence SEQ ID NO 002; and/or
   c. a peptide linker connecting the HLA heavy chain polypeptide to the IgG Fc polypeptide, particularly a peptide linker between 5 and 20 amino acids in length, more particularly a peptide linker with the sequence SEQ ID NO 003;
Item 7. The combination medicament for use according to any one of the items 1 to 6, wherein the HLA fusion protein comprises, or essentially consists of, the sequence designated SEQ ID NO 005.
Item 8. The combination medicament for use according to any one of the items 1 or 7, wherein the HLA fusion protein comprises a secretory signal, particularly wherein the secretory signal is 16 to 30 amino acids in length, more particularly wherein the secretory signal is removed by cleavage during the process of secretion from the cell, still more particularly wherein the secretory signal has the sequence SEQ ID NO 004.
Item 9. The combination medicament for use according to any one of the items 1 to 8, wherein the HLA fusion protein is in the form of a dimer, said dimer comprising, or essentially consisting of a first HLA monomer and a second HLA monomer;
   wherein the first HLA monomer essentially consists of a first HLA fusion protein as specified in any one of the items 1 to 8, and a first β2m polypeptide; and
   wherein the second HLA monomer essentially consists of a second HLA fusion protein as specified in any one of the items 1 to 8, and a second β2m polypeptide;
particularly wherein the first and the second HLA monomer are identical.
Item 10. An inhibitor of the interaction between CD47 and SIRPα for use in the treatment of a malignant neoplastic disease, wherein the inhibitor of the interaction between CD47 and SIRPα for use in the treatment of a malignant neoplastic disease is administered prior to, in combination with, or subsequent to, an HLA fusion protein as specified in any one of the items 1 to 9.
Item 11. The combination medicament for use according to any one of the items 1 to 8, or the inhibitor of the interaction between CD47 and SIRPα for use according to item 10, wherein said inhibitor of the interaction between CD47 and SIRPα binds to CD47 or SIRPα with a dissociation constant of at least 10⁻⁷ M⁻¹.
Item 12. The combination medicament for use according to any one of the items 1 to 8, or 11, or the inhibitor of the interaction between CD47 and SIRPα for use according to item 10 or 11, wherein said inhibitor of the interaction between CD47 and SIRPα comprises, or essentially is, an antibody, an antibody fragment, or an antibody-like molecule.
Item 13. The combination medicament for use according to any one of the items 1 to 8, 11 or 12, or the inhibitor of the interaction between CD47 and SIRPα for use according to any one of the items 10 to 12, wherein the inhibitor of the interaction between CD47 and SIRPα comprises, or consists of, a fusion protein, said fusion protein comprising:
   a SIRPα polypeptide extracellular domain; and
   an Ig fc domain;
   particularly wherein the inhibitor of the interaction between CD47 and SIRPα is selected from ALX148, TTI-622, or TTI-621.
Item 14. The combination medicament for use according to any one of the items 1 to 8, or 11 to 13, or the inhibitor of the interaction between CD47 and SIRPα for use according to any one of the items 10 to 13, wherein said HLA fusion protein, or said inhibitor of the interaction between CD47 and SIRPα, is provided in a dosage form suitable for systemic delivery.
Item 15. The combination medicament for use according to any one of the items 1 to 8, or 11 to 14, or the inhibitor of the interaction between CD47 and SIRPα for use according to any one of the items 10 to 14, wherein the malignant neoplastic disease is
   a. a blood-cell derived cancer, myeloma, or
   b. a solid tumor, particularly breast cancer, more particularly a form of ductal cell carcinoma.

The invention is further illustrated by the following examples and figures, from which further embodiments and advantages can be drawn. These examples are meant to illustrate the invention but not to limit its scope.

### DESCRIPTION OF THE FIGURES

FIG. 1 (A) shows how the interactions of SIRPα and LILRB2 on macrophages engage with their natural ligands on tumor cells, resulting in immunosuppressive signals. (B) shows summary of experimental design testing whether releasing the brakes on macrophages killing of cancer cells by blocking LILRB1/2 with HLA fusion protein candidate iosH2 may be combined with blocking SIRPα/CD47 axis with antibodies or natural ligands to enhance tumoricidal activity.

FIG. 2 shows superior cell viability and HLA fusion protein expression properties of the HLA-B57^{(A46E/V97R)} IgG4 fusion protein. Fusion protein expression from clones transfected with HLA B57.β2m (DGC8-T39, DGC8-T64, & DGC8-73) and HLA-B57^{(A46E/V97R)}.β2m (DGC8-T54, DGC8-T75 & DGC8-91) on the basis of cell viability (A) and expressed protein titers (B). Table summarizes yield at the different transfection ratios tested. Equivalent RNA profiles of the (C) HLA-B57^{(A46E/V97R)} IgG4 fusion protein and (D) β2m expressed from vectors within CHO cell clones.

FIG. 3 shows quantitative estimation of the binding affinities of LILRB2 with non-β2m-associated HLA-B57 or HLA-B57^{(A46E/V97R)} fusion proteins, and HLA-B57^{(A46E/V97R)}. β2m measured by ELISA. β2m-free HLA-B57 has an EC50 of 21 nM, HLA-B57^{(A46E/V97R)} EC50 of 8.3 nM, & HLA-B57^{(A46E/V97R)}. 32m EC50 5.72 nM, demonstrating that amino acid substitutions do not reduce the binding of HLA-B57 heavy chain to LILRB2.

FIG. 4 shows increased phagocytosis of (A) a liquid cancer, using RPMI8226 multiple myeloma cells, and (B) a solid cancer, using BT474 breast cancer cells using HLA-B57^{(A46E/V97R)}.β2m (iosH2) in combination with anti-SIRPα or anti-CD47 antibodies. Cancer cell lines derived from solid and liquid tumors were co-cultured with human primary macrophages and phagocytosis was measured for 36 hours in IncuCyte live-cell imaging system. Experiments were repeated using at least 2 biologically independent samples. IgG1 10 μg/ml, IgG4 20 μg/ml, iosH2 20 μg/ml, anti-CD47 antibody 10 μg/ml, MK4830 10 μg/ml, anti-SIRPα antibody 10 μg/ml. Error bars, SEM of n=2 biological replicates with each containing 2 technical replicates. Statistical analysis was performed using two-way repeated measures ANOVA, followed by Dunnett's post-hoc analysis, *p<0.05, ** p<0.01, *** p<0.001, **** p<0.0001

### EXAMPLES

### Example 1. Generation of HLA Fusion Proteins

In previous work by the inventors, an HLA fusion protein (iosH1) was designed by linking the heavy chain extracellular domain of the HLA-B57:01:01 polypeptide to an IgG4 Fc polypeptide (SEQ ID NO 002). To increase the yield of this HLA fusion protein, inhibitory amino acids identified in the natural HLA-B57 extracellular domain amino acid sequence were altered by substitution of an alanine (A) residue at position 46 to glutamine (E), and a valine (V) at position 97 to an arginine (R), providing a variant HLA-B57 polypeptide (SEQ ID NO 001). This was fused to the IgG4 polypeptide via a linking peptide (SEQ ID NO 003), to provide a variant HLA-B57 fusion protein (SEQ ID NO 005, losH2). cDNA encoding the recombinant HLA-B57^{(A46E/V97R)} fusion protein and the natural HLA-B57-derived fusion protein control, lacking the two mutations, were cloned into commercial expression vectors (Probiogen) downstream of a nucleic acid sequence encoding a secretion signal (SEQ ID NO 004). The vector constructs expressing HLA-B57-Fc & HLA-B57^{(A46E/V97R)}-Fc were co-transfected into Chinese hamster ovary (CHO) cells along with a plasmid comprising a nucleic acid encoding the β2m protein (SEQ NO 006) by microporation (MP) using the NEON Transfection Kit (Life Technologies #MPK10096). CHO-DG44 starter cells were transfected at different ratios of HLA fusion protein to β2m plasmid (4:1, 2:1, 1:1, 1:2). Selected clone pools were grown in standardized shaker flasks and with a defined cell seeding density of 4E5 vc/mL in 125 mL of PBG-CD-C4 supplemented medium including puromycin and methotrexate. Following adjusted selection pression with antibiotics, individual clone pools were selected for analysis. Measurement of viabilities and viable cell densities were performed using the Vi-CELL XR System, and Trypan blue cell exclusion method. Titer quantifications were measured at different time points (days) using an Octet RED machine (ForteBio, a Pall Division) with Protein A biosensors.

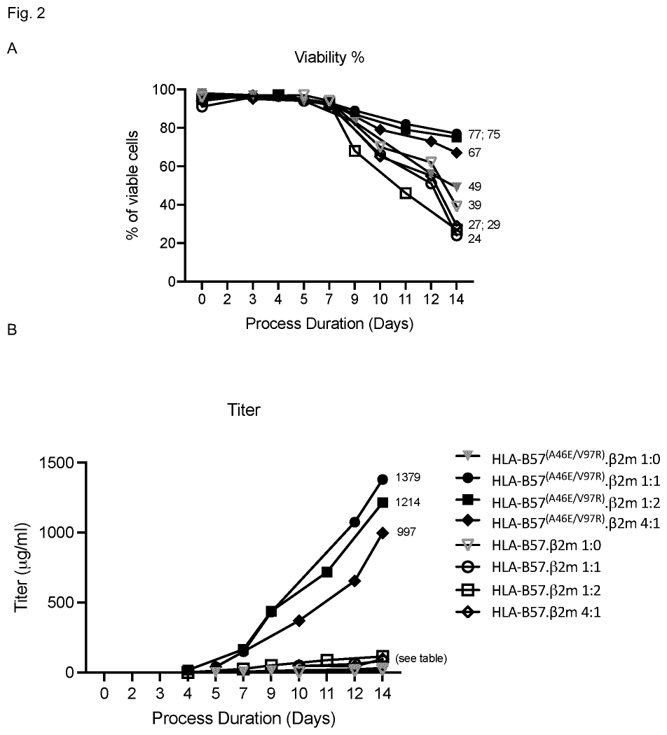

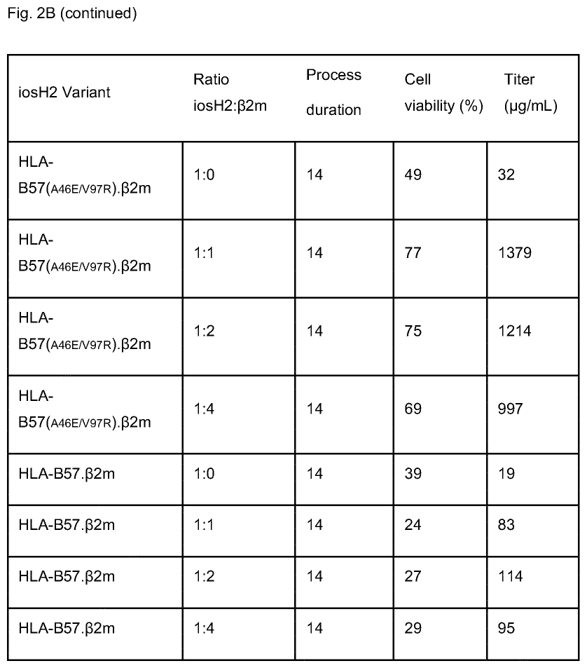

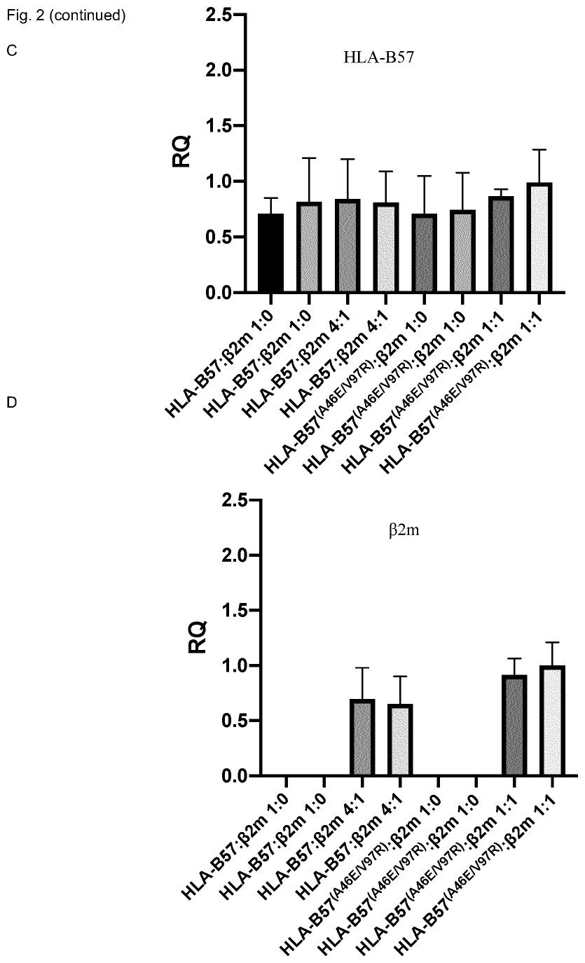

Analysis of clones expressing the HLA-B57 or variant fusion protein demonstrated that wildtype HLA-B57.β2m cell viability and titers are significantly lower than HLA-B57^{(A46E/V97R)}. β2m (FIG. 2A and B). Equimolar RNA levels of the natural HLA-B57or altered HLA-B57^{(A46E/V97R)} relative to β2m were confirmed in selected clones cell clones (FIG. 2C and D), suggesting the amino acid modifications drove increased expression.

The HLA-B57^{(A46E/V97R)}. β2m complex was then isolated from filtered CHO cell supernatants by affinity column purification. Purification of proteins and removal of β2m under acidic conditions was performed as a two-step purification protocol. As a first step, Protein G Sepharose [(4 Fast Flow) Sigma, #GE-17-0618-01)] beads were used to capture HLA-B57^{(A46E/V97R)} associated with β2m from supernatants. After an overnight incubation at 4 degrees on a rocker, the recovered beads were washed in PBS, and subsequently HLA-B57^{(A46E/V97R)} fusion proteins, in which each HLA polypeptide was still associated with β2m were eluted using standard IgG-Elution Buffer (pH 2.8) (Pierce™ IgG Elution Buffer, Thermo Fischer #21004). A second step of size exclusion chromatography-based purification was performed to separate HLA-B57^{(A46E/V97R)} from β2m under acidic conditions, to provide a non-β2m-associated HLA fusion protein. A Superdex 10/300 gel filtration column, pre-equilibrated in Sodium Citrate (100 mM, pH 3.0) was used for the separation. An injection of 0.5 ml of the protein at 2.0 mg/ml concentration was applied, and the desired HLA-B57^{(A46E/V97R)} protein peak eluted at 12.7 ml and the peak for β2m eluted at 22.0 ml.

### Example 2. Quantification of the Interaction of LILRB2 with Non-.β2m-Associated or .β2m Associated HLA Fusion Proteins

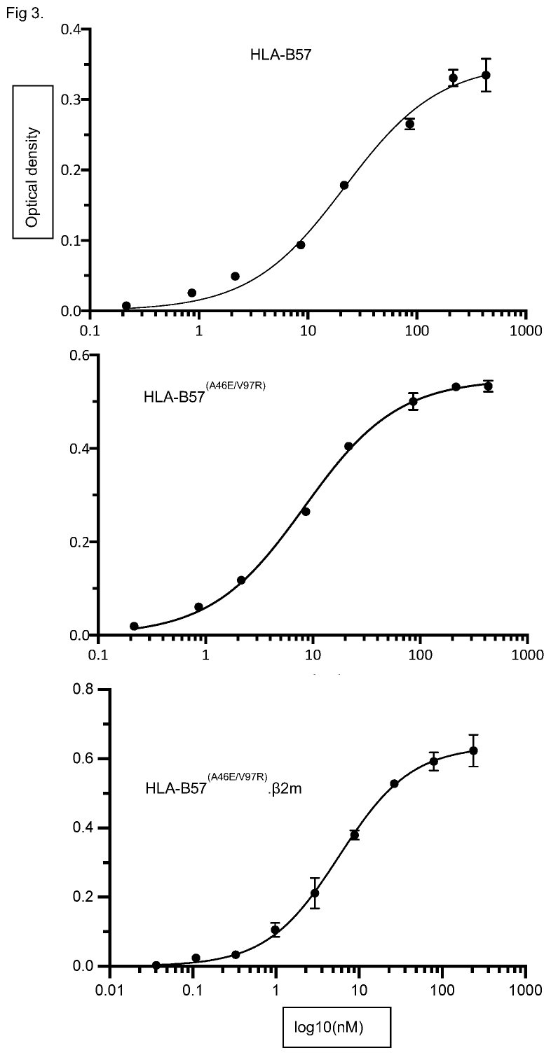

The inventors went on to dissect the immunological properties most relevant to tumor immunity associated with HLA-B57^{(A46E/V97R)} formats, all lacking association with a peptide epitope associated with the peptide-binding groove of the HLA class I domain of the fusion protein. The affinity of interaction of LILRB2 with wildtype HLA-B57 fusion protein (dimers) lacking β2m, HLA-B57^{(A46E/V97R)} fusion protein (dimers) lacking β2m, and HLA-B57^{(A46E/V97R)} fusion protein dimers still bound to β2m was measured using the enzyme-linked immunosorbent assay (ELISA) method. Flat bottom Pierce™ Streptavidin coated high binding capacity 96 well plates (Pierce #15500) were coated with 50 μl of c-terminally biotinylated antigen molecules (LILRB2, BPS Bioscience #100335) immobilized at a final concentration of 5 μg/ml in PBS buffer. PBS and IgG isotype were used as negative controls. A serial dilution of HLA-B57, or HLA-B57^{(A46E/V97R)} lacking β2m, and HLA-B57^{(A46E/V97R)}.β2m (eight concentration points: 10, 2.5, 1, 0.25, 0.1, 0.025, 0.01, 0.0025 μg/ml) was applied (50 μl) in duplicates. An APC conjugated goat anti-human IgG antibody (Jackson Immuno Research #109-135-098) with 1:100 dilution in TBS (50 μl) was used for detection. Finally, 50 μl TBS in each well was added and a fluorescence scan was performed with APC excitation and emission wavelengths of 650 nm & 660 nm, respectively. Graphpad Prism v9.1.2 and the three-parameter based log (agonist) vs. response model was used to determine the EC50 of the interaction. Improved binding of the variant HLA-B57 fusion protein to LILRB2 was observed, either as an HLA-B57^{(A46E/V97R)} lacking association with β2m, and particularly when associated with β2m, suggesting the variant has high immunomodulatory potential (FIG. 3).

### Example 3 Increased Killing of Tumor Cells In Vitro

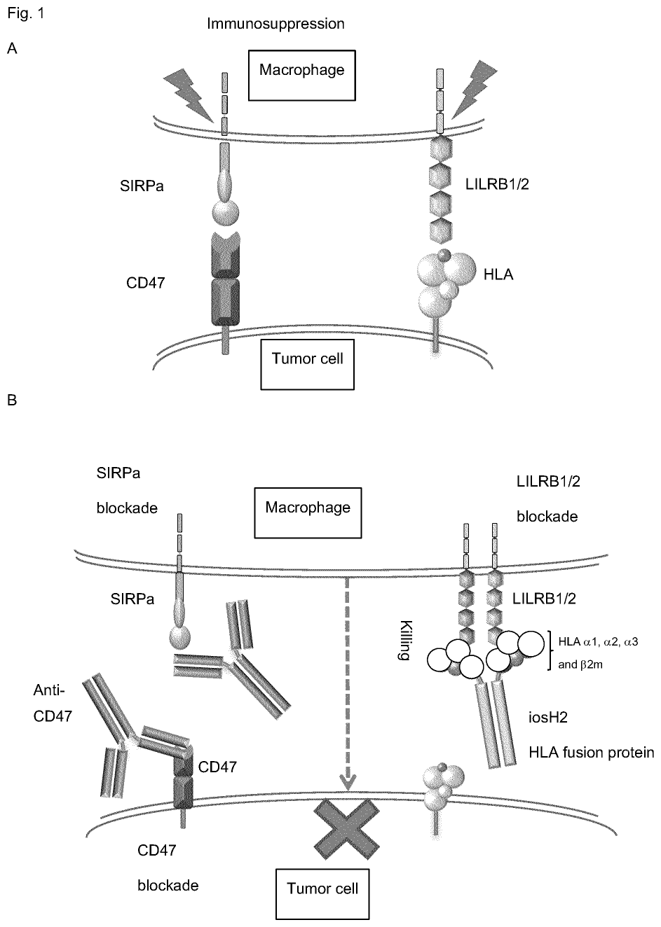

Next, the capacity of the β2m associated HLA-B57^{(A46E, V97R)} IgG4 Fc fusion protein (termed iosH2) to induce phagocytosis of tumor cells by human primary macrophages was assessed towards liquid (RPMI8226 multiple myeloma, DSMZ Cat. #ACC402) and solid (BT474 breast ductal carcinoma, DSMZ Cat. #ACC64) cancer cells, in comparison to PBS, IgG1 (Biolegend; Cat. #403502) and IgG4 (Biolegend; Cat. #403702) isotype controls and a benchmark anti-LILRB2 antibody (MK4830) (produced in house based on sequence in US 2018/0298096 A1). In addition, to explore whether combined inhibition of two pathways that modulate phagocyte activation can enhance the effect of iosH2 (FIG. 1), simultaneous treatment with either an anti-CD47 antibody (LubioScience; Cat. #BE0019-1) or an anti-SIRPα antibody (in house produced based on sequence in WO 2020068752 A1, heavy chain of SEQ ID NO 007 and light chain of SEQ ID NO 008) was assessed.

Cancer cell lines derived from the indicated liquid and solid tumors were co-cultured with human primary macrophages, and phagocytosis of tumor cells was monitored according to the manufacturer's instructions for 36 hours using IncuCyte incubator and live-cell imaging system (Vitaris AG, MCO-230AICUVH-PE). Cancer cells were stained with CellTrace™ CFSE (ThermoFisher) according to manufacturer's instructions and subsequently 1000 cells/well were plated in flat-bottom 96 well plates (Greiner) together with 1000-5000 primary T cells. Media contained 250 nM of Cytotox Red (Sartorius). Live cell imaging was performed using the Incucyte S3 Live-Cell Analysis System (Sartorius). 4 non-adjacent images per well were analyzed with IncuCyte software v2020C. The CFSE signal was segmented in green objects, and every object was counted as cancer cell. Dead cells were identified with Cytotox signal segmented in red objects. Cancer cell death was detected by colocalization of green and red objects.

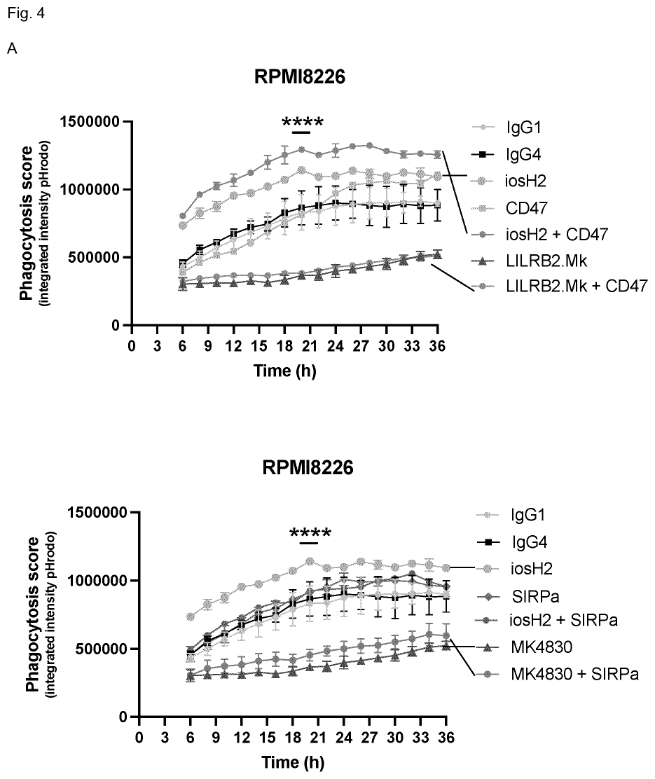

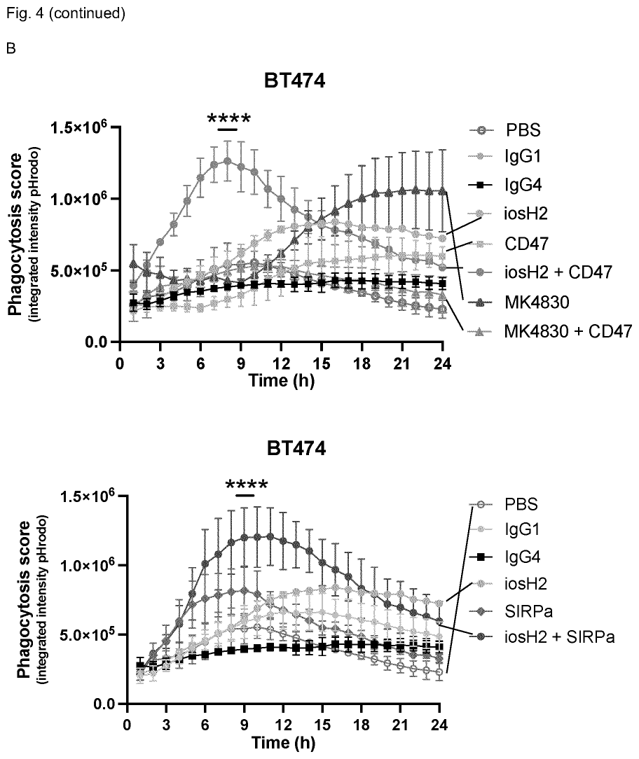

The results in FIG. 4 demonstrate that HLA-B57^{(A46E, V97R)}. β2m (iosH2) significantly and rapidly increased phagocytosis of macrophages already 3 hours after co-culturing. Although the rate and dynamic of phagocytosis was different for different cell lines, the effect of iosH2 was not dependent on the type of cancer cell. losH2 stimulation was also superior to the effect induced by benchmark anti-LILRB2 antibody MK4830, which provoked delayed phagocytosis of breast cancer, but had no effect on myeloma cells. Anti-CD47 antibody, or anti-SIRPα antibody had no effect on myeloma phagocytosis rate as single agent drug; however, a combination of iosH2 with anti-CD47 antibody led to an elevated myeloma phagocytosis rate, which was not observed with combined anti-CD47 antibody and MK4830. For the breast cancer cell line BT474, anti-CD47 antibody also enhanced losH2 induced phagocytosis, despite have little effect alone. Anti-SIRPα antibody together with iosH2 showed the highest rates of phagocytosis compared to all tested mono and combination therapies in breast cancer, however, it had no effect in treating the myeloma cell line.

Sequences:
variant HLA-B57: 01 extracellular domain A46E V97R (artificial)
SEQ ID NO 001
GSHSMRYFYTAMSRPGRGEPRFIAVGYVDDTQFVRFDSDAASPRMEPRAPWIEQEGPEYWDGE
TRNMKASAQTYRENLRIALRYYNQSEAGSHIIQRMYGCDVGPDGRLLRGHDQSAYDGKDYIAL
NEDLSSWTAADTAAQITQRKWEAARVAEQLRAYLEGLCVEWLRRYLENGKETLQRADPPKTHV
THHPISDHEATLRCWALGFYPAEITLTWQRDGEDQTQDTELVETRPAGDRTFQKWAAVVVPSG
EEQRYTCHVQHEGLPKPLTLRWEPSSQS
Optimized IgG4 Fc
SEQ ID NO 002
ESKYGPPCPPCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLG
peptide linker (artificial)
SEQ ID NO 003
GGGGSGGGGS
secretory signal (artificial)
SEQ ID NO 004
AAAMNFGLRLIFLVLTLKGVQC
variant HLA-B57: 01 extracellular domain A46E V97R IgG4 fusion
protein
(artificial)
SEQ ID NO 005
GSHSMRYFYTAMSRPGRGEPRFIAVGYVDDTQFVRFDSDAASPRMEPRAPWIEQEGPEYWDGE
TRNMKASAQTYRENLRIALRYYNQSEAGSHIIQRMYGCDVGPDGRLLRGHDQSAYDGKDYIAL
NEDLSSWTAADTAAQITQRKWEAARVAEQLRAYLEGLCVEWLRRYLENGKETLQRADPPKTHV
THHPISDHEATLRCWALGFYPAEITLTWQRDGEDQTQDTELVETRPAGDRTFQKWAAVVVPSG
EEQRYTCHVQHEGLPKPLTLRWEPSSQSGGGGSGGGGSESKYGPPCPPCPAPEFLGGPSVFLF
PPKPKDTLMISRTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHEAL
HNHYTQKSLSLSLG
Variant HLA-B57 extracellular domain,
peptide linker (underlined), IgG4 Fc (italics)
beta-2-microglobulin (homo sapiens)
SEQ ID NO 006
IQRTPKIQVYSRHPAENGKSNFLNCYVSGFHPSDIEVDLLKNGERIEKVEHSDLSFSKDWSFY
LLYYTEFTPTEKDEYACRVNHVTLSQPKIVKWDRDM.
SEQ ID NO 007
anti-SIRP alpha antibody heavy chain (artificial)
QVQLVQSGAEVKKPGASVKVSCKASGYTFRGYGISWVRQAPGQGLEWMGWISAYGGETNYAQK
LQGRVTMTTDTSTSTAYMELRSLRSDDTAVYYCAREAGSSWYDFDLWGRGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
anti-SIRP alpha antibody light chain
(artificial)
SEQ ID NO 008
DIQMTQSPSSVSASVGDRVTITCRASQGISSWLAWYQQKPGKAPKLLIYAASNLQSGVPSRFSG
SGSGTDFTLTISSLQPEDFATYYCQQGASFPITFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC
HLA-CW0802 fusion protein (artificial)
SEQ ID NO 009
CSHSMRYFYTAVSRPGRGEPRFIAVGYVDDTQFVQFDSDAASPRGEPRAPWVEQEGPEYWDR
ETQKYKRQAQTDRVSLRNLRGYYNQSEAGSHTLQRMYGCDLGPDGRLLRGYNQFAYDGKDYI
ALNEDLRSWTAADKAAQITQRKWEAAREAEQRRAYLEGTCVEWLRRYLENGKKTLQRAEHPKT
HVTHHPVSDHEATLRCWALGFYPAEITLTWQRDGEDQTQDTELVETRPAGDGTFQKWAAVVVP
SGEEQRYTCHVQHEGLPEPLTLRWGPSSQPGGGGSGGGGSESKYGPPCPPCPAPEFLGGPSVF
LFPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSRWQEGNVFSCSVMHE
ALHNHYTQKSLSLSLG
HLA-CW0802 extracellular domain, peptide linker (underlined),
IgG4 Fc (italics)
HLA-B58 fusion protein
(artificial)
SEQ ID NO 010
GSHSMRYFYTAMSRPGRGEPRFIAVGYVDDTQFVRFDSDAASPRTEPRAPWIEQEGPEYWDGE
TRNMKASAQTYRENLRIALRYYNQSEAGSHIIQRMYGCDLGPDGRLLRGHDQSAYDGKDYIAL
NEDLSSWTAADTAAQITQRKWEAARVAEQLRAYLEGLCVEWLRRYLENGKETLQRADPPKTHV
THHPVSDHEATLRCWALGFYPAEITLTWQRDGEDQTQDTELVETRPAGDRTFQKWAAVVVPSG
EEQRYTCHVQHEGLPKPLTLRWEPSSQSGGGGSGGGGSESKYGPPCPPCPAPEFLGGPSVFLF
PPKPKDTLMISRTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHEAL
HNHYTQKSLSLSLG
HLA-B58: 01 extracellular domain, peptide linker (underlined),
IgG4 Fc (italics)
HLA-A30: 01 fusion protein
(artificial)
SEQ ID NO 011
GSHSMRYFSTSVSRPGSGEPRFIAVGYVDDTQFVRFDSDAASQRMEPRAPWIEQERPEYWDQ
ETRNVKAQSQTDRVDLGTLRGYYNQSEAGSHTIQIMYGCDVGSDGRFLRGYEQHAYDGKDYI
ALNEDLRSWTAADMAAQITQRKWEAARWAEQLRAYLEGTCVEWLRRYLENGKETLQRTDPPK
THMTHHPISDHEATLRCWALGFYPAEITLTWQRDGEDQTQDTELVETRPAGDGTFQKWAAVV
VPSGEEQRYTCHVQHEGLPKPLTLRWELSSQPGGGGSGGGGSESKYGPPCPPCPAPEFLGGP
SVFLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFS
CSVMHEALHNHYTQKSLSLSLG
HLA-A30: 01 extracellular domain, peptide linker (underlined),
IgG4 Fc (italics)

### CITATIONS

Colonna M. et al. 1997 J. Exp. Med. 186 (1): 1809
Ring et al., 2017, Proc Natl Acad Sci 114 (49): E10578-E10585),
WO 2017153438 A1; WO2016124661A1; WO2018 029284 A1; WO 2019/023347; WO 2013/056352; WO 2018/190719; US 2010/0239579

All scientific publications and patent documents cited in the present specification are incorporated by reference herein.

1. A combination medicament comprising:
a. a soluble human leukocyte antigen (HLA) heavy chain polypeptide, particularly wherein the HLA heavy chain polypeptide is selected from:
an extracellular domain of an HLA heavy chain selected from HLA-B57, HLA-C08, HLA-A25, HLA-B58, HLA-B27, HLA-A30, HLA-B53, or HLA-C12; or
a variant of said extracellular domain of an HLA heavy chain, wherein said variant is characterized by a sequence similarity of at least (≥) 95%, particularly ≥98%, to an extracellular domain of an HLA heavy chain selected from HLA-B57, HLA-C08, HLA-A25, HLA-B58, HLA-B27, HLA-A30, HLA-B53, or HLA-C12, and a similar biological activity to the respective HLA heavy chain; and
b. an inhibitor of the interaction between CD47 and SIRPα.

2. The combination medicament according to claim 1, wherein the HLA heavy chain polypeptide comprises, particularly consists of, an HLA fusion protein comprising
an HLA heavy chain domain selected from:
an extracellular domain of an HLA heavy chain, particularly an HLA heavy chain selected from HLA-B57, HLA-C08, HLA-A25, HLA-B58, HLA-B27, HLA-A30, HLA-B53, or HLA-C12; or
a variant of said extracellular domain of an HLA heavy chain, wherein said variant is characterized by a sequence similarity of at least (≥) 95%, particularly ≥98%, and a similar biological activity to the respective HLA heavy chain; and
an immunoglobulin crystallizable fragment (Ig Fc) polypeptide, particularly an IgG Fc polypeptide, more particularly an IgG4 Fc polypeptide.

3. The combination medicament according to claim 1, wherein the HLA heavy chain polypeptide is associated with a β2m polypeptide.

4. The combination medicament according to claim 3, wherein the HLA heavy chain polypeptide is non-covalently associated with the β2m polypeptide at a ratio of between 3:5 to 7:5, more particularly between 4:5 to 6:5, more particularly at a ratio of 1:1.

5. The combination medicament according to claim 1, wherein the HLA heavy chain polypeptide, or the HLA heavy chain domain is a variant of the extracellular domain of HLA-B57, and wherein the variant of the extracellular domain of HLA-B57 is characterized by an E at position 46, and an R at position 97.

6. The combination medicament according to claim 1, wherein the HLA heavy chain polypeptide, or the HLA heavy chain domain comprises, or essentially consists of the sequence SEQ ID NO 001.

7. The combination medicament according to claim 2,
wherein the HLA fusion protein comprises an IgG Fc polypeptide, particularly an IgG4 Fc polypeptide, more particularly an IgG4 Fc polypeptide with the sequence SEQ ID NO 002;
and wherein a peptide linker connects the HLA heavy chain domain to the IgG Fc polypeptide, particularly a peptide linker between 5 and 20 amino acids in length, more particularly a peptide linker with the sequence SEQ ID NO 003.

8. The combination medicament according to claim 2, wherein the HLA heavy chain domain is positioned N-terminal relative to the Ig Fc polypeptide.

9. The combination medicament according to claim 2, wherein the HLA fusion protein comprises the sequence designated SEQ ID NO 005.

10. The combination medicament according to claim 2, wherein the HLA fusion protein comprises a secretory signal, particularly wherein the secretory signal is 16 to 30 amino acids in length, more particularly wherein the secretory signal is removed by cleavage during the process of secretion from the cell, still more particularly wherein the secretory signal has the sequence SEQ ID NO 004.

11. The combination medicament according to claim 2, wherein the HLA fusion protein is in the form of a dimer, said dimer comprising, or essentially consisting of a first HLA monomer and a second HLA monomer;
wherein the first HLA monomer essentially consists of a first HLA fusion protein, and a first β2m polypeptide; and
wherein the second HLA monomer essentially consists of a second HLA fusion protein, and a second β2m polypeptide;
particularly wherein the first and the second HLA monomer are identical.

12. The combination medicament according to claim 1, wherein the HLA heavy chain polypeptide is not associated with a peptide epitope.

13. A method for treating a cancer comprising administering to a subject in need thereof an inhibitor of the interaction between CD47 and SIRPα,
wherein the inhibitor of the interaction between CD47 and SIRPα is administered prior to, in combination with, or subsequent to, a soluble HLA heavy chain polypeptide as specified in claim 1,
thereby treating the cancer.

14. The combination medicament according to claim 1, wherein said inhibitor of the interaction between CD47 and SIRPα binds to CD47 or SIRPα with a dissociation constant of 10⁻⁷ mol/L or lower (higher affinity).

15. The combination medicament according to claim 1, wherein said inhibitor of the interaction between CD47 and SIRPα comprises, or essentially is, an antibody, an antibody fragment, or an antibody-like molecule;
particularly wherein the antibody is selected from the group consisting of Hu5F9-G4, TI-061, IBI188, CC-90002, lemzoparlimap/TJC4, SL-172154, IMC-002, GS-0189, and ADU1805.

16. The combination medicament according to claim 1, wherein the inhibitor of the interaction between CD47 and SIRPα comprises, or consists of, a fusion protein, said fusion protein comprising:
a SIRPα polypeptide extracellular domain; and
an Ig fc domain;
particularly wherein the inhibitor of the interaction between CD47 and SIRPα is selected from ALX148, TTI-622, or TTI-621.

17. A method for treating cancer comprising administering to a subject in need thereof a combination medicament according to claim 1, thereiby treating the cancer.

18. The method according to claim 17, wherein the cancer is a solid tumour, particularly a carcinoma.

19. The method according to claim 17, wherein the cancer is a liquid cancer, particularly a leukemia, lymphoma or myeloma.

20. The method according to claim 17, wherein said soluble HLA heavy chain polypeptide, or said inhibitor of the interaction between CD47 and SIRPα is provided in a dosage form suitable for systemic delivery.

21. The method according to claim 17, wherein the cancer is a solid tumor, particularly breast cancer, more particularly a form of ductal cell carcinoma.


SEQUENCE LISTING (11 sequences)

SEQ ID NO: 1 - protein, 280 aa, synthetic construct
GSHSMRYFYTAMSRPGRGEPRFIAVGYVDDTQFVRFDSDAASPRMEPRAPWIEQEGPEYWDGETRNMKASAQTYRENLRIALRYYNQSEAGSHIIQRMYGCDVGPDGRLLRGHDQSAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQS

SEQ ID NO: 2 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 10 aa, synthetic construct
GGGGSGGGGS

SEQ ID NO: 4 - protein, 22 aa, synthetic construct
AAAMNFGLRLIFLVLTLKGVQC

SEQ ID NO: 5 - protein, 518 aa, synthetic construct
GSHSMRYFYTAMSRPGRGEPRFIAVGYVDDTQFVRFDSDAASPRMEPRAPWIEQEGPEYWDGETRNMKASAQTYRENLRIALRYYNQSEAGSHIIQRMYGCDVGPDGRLLRGHDQSAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 99 aa, Homo sapiens [sequence omitted]

SEQ ID NO: 7 - protein, 450 aa, synthetic construct
QVQLVQSGAEVKKPGASVKVSCKASGYTFRGYGISWVRQAPGQGLEWMGWISAYGGETNYAQKLQGRVTMTTDTSTSTAYMELRSLRSDDTAVYYCAREAGSSWYDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, synthetic construct
DIQMTQSPSSVSASVGDRVTITCRASQGISSWLAWYQQKPGKAPKLLIYAASNLQSGVPSRFSGSGSGTDFTLTISSLQPEDFATYYCQQGASFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 518 aa, synthetic construct
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 518 aa, synthetic construct
GSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSSQSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 518 aa, synthetic construct
GSHSMRYFSTSVSRPGSGEPRFIAVGYVDDTQFVRFDSDAASQRMEPRAPWIEQERPEYWDQETRNVKAQSQTDRVDLGTLRGYYNQSEAGSHTIQIMYGCDVGSDGRFLRGYEQHAYDGKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG